(19) United States
(12) Patent Application Publication
(10) Pub. No.: US 2009/0017089 A1
(43) Pub. Date: Jan. 15, 2009

(54) Multiple active agents such as anthelmintics sustained release delivery device

(76) Inventors: David Malcolm Leathwick, Palmerston North (NZ); Rex Munday, Hamilton (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 10/576,589

(22) PCT Filed: Oct. 26, 2004
(86) PCT No.: PCT/NZ2004/000267

§ 371 Date: Sep. 5, 2006

(30) Foreign Application Priority Data
Oct. 24, 2003 (NZ) 529177

(51) Int. Cl. A61K 9/00; A61K 31/4184; A61P 33/00

(52) U.S. Cl. 424/423; 514/394

(57) ABSTRACT

A method of treating animals characterised by the step of introducing to the animal a single delivery device containing two or more active agents, wherein the delivery device is configured to release an effective amount of active agents over a defined time period of 3-14 days.

### TECHNICAL FIELD

This invention relates to a process for the administration of active agents to animals.

In particular, this invention relates to a process for administering an anthelmintic preparation combining two or more anthelmintics to an animal to reduce the level of parasitic infestation and in a manner designed to achieve increased efficacy against resistant worms.

### BACKGROUND ART

Parasites are a major production-limiting factor in livestock grazing systems throughout the world. The size of the issue can be gauged by the global market for parasiticides which is approximately US $3 billion annually of which nearly US $2 billion goes to production animals (sheep, cattle, poultry and pigs). The cattle market alone is nearly US $900M per annum.

The size of the market for parasiticides also reflects the fact that throughout the world most production systems rely heavily on the use of anthelmintic drugs to control infections in livestock. However, in some countries this dependence on the use of anthelmintics is now threatened by the development of resistance amongst parasite populations.

Countries such as South Africa, Australia and parts of South America already have serious resistance problems in parasites of sheep and goats. New Zealand, Great Britain and France also have significant and developing problems. In Australia, for example, almost every sheep farm has resistance to at least one “action-family” of anthelmintic. Survey results indicate that on more than 90% of Australian sheep farms at least 2 action-families (the benzimidazole and Levamisole groups) are less than fully effective due to resistance. Furthermore, the last 2-3 years has seen a rapid increase in the prevalence of resistance to the 3^{rd} action-family, the macrocyclic lactone (ML) group.

By comparison, New Zealand has resistance on about 60% of sheep farms, but most still have effective use of at least 2 action-families. The problem does, however, continue to worsen and recent years have seen confirmation of ML resistance in sheep flocks. In addition, New Zealand has an unquantified but significant problem with ML resistance in parasites of cattle.

In response to the threat posed by anthelmintic resistance, there has been substantial research into the factors associated with its development and means of preventing or delaying it.

Genetic theory, reinforced by a number of modelling studies (Tabashnik & Croft, 1982. Environmental Entomology 11, 1134-1137; Barnes et al. 1995. Parasitology Today 11, 56-63; Leathwick & Sutherland 2002. Proceedings of the 32^{nd} Seminar, The Society of Sheep and Beef Cattle Veterinarians, N.Z. Veterinary Association, 115-127) shows that the efficacy of an anthelmintic against resistant worms, in particular the heterozygotes (i.e., worms carrying one resistant and one susceptible allele or gene) can have a substantial effect on the rate at which resistance develops.

When resistant genes are rare in the population, mating probabilities determine that they nearly always occur in the heterozygote form. Hence if the anthelmintic kills all or most heterozygotes then it will be very difficult for resistance to build up in the population. If, however, the anthelmintic does not kill the heterozygote worms, they will build up in the population, and interbreeding will produce even more resistant homozygotes. In essence, the efficacy of any anthelmintic product against resistant worms is a key factor in delaying the development of severe, production-limiting resistance.

It was shown as early as 1978 (Prichard et al. 1978. Veterinary Parasitology 4, 309-315) that extending the period over which worms are exposed to benzimidazole drenches increases their efficacy. A number of subsequent studies have confirmed that not only can efficacy be increased by this approach but also that the daily dose required to do this can be lower than that required for a single dose (Le Jambre et al. 1981. Research in Veterinary Science, 31, 289-294; Sangster et al. 1991. Research in Veterinary Science 51, 258-263). The same principle has been shown to apply to the ML class of anthelmintics, but not to Levamisole.

Repetitive dosing of extensively-grazed animals with anthelmintics is not practicable. A controlled release device, which would permit extended drug exposure with only a single administration, is therefore required.

Some controlled release devices for anthelmintics are presently on the market. The controlled release capsules (CRCs) made by CapTec release between ⅕^{th} and 1/10^{th} the normal therapeutic dose (depending on animal liveweight) of either albendazole or ivermectin for 100 days.

Currently-available oral products provide albendazole at doses of approximately 3.8 to 5 mg/kg and the macrocyclic lactones at approximately 0.2 mg/kg. Doses given by current CRC's are much lower, being of the order of 0.5-1.0 mg/kg/day for albendazole and 0.02-0.04 mg/kg/day for the macrocyclic lactones.

Although Le Jambre et al, 1981 (Research in Veterinary Science, 31, 289-294) showed increased efficacy by releasing a benzimidazole at low doses in a short duration CRC the commercially available albendazole CRCs appear to show no better efficacy against established drug-susceptible Ostertagia (<95%) than would be expected from a single oral dose at 5 mg/kg (Anderson et al. 1988. Australian Advances in Veterinary Science, 60-61).

Similar studies in NZ suggest that efficacy against some worm species is no better than a single dose (Extender 100™ Technical manual). Barger, 1993 (Proceedings 23^{rd} Seminar, Sheep and Beef Cattle Society, New Zealand Veterinary Association, 129-136) claims that while the efficacy of the CRC against resistant worms is variable, it is superior to that of a single oral drench. However, other studies do not support this view (Macchi et al. 2001. New Zealand Veterinary Journal 49, 48-53; Leathwick, et al. 2001. New Zealand Veterinary Journal, 50(2): 70-76). Thus it remains unclear whether the 100 day albendazole capsule (Extender™) does in fact confer increased efficacy against established resistant worms.

Further, the efficacy of the 100 day ivermectin CRC (Maximiser™) against resistant adult worms has been shown to be no better than the standard single oral dose of ivermectin (Sutherland et al., 2002. Veterinary Parasitology, 109: 91-99).

Thus, despite having known for 25 years that extending the duration of worm exposure to some actives can substantially increase efficacy there has never been a product produced which fully capitalises on this knowledge. Further, the only product on the market for sheep and cattle (NZ & Australia at least) lasts for 100 days, resulting in long withholding periods for the ivermectin variant.

Furthermore, the prolonged delivery of small doses of anthelmintic may actually select for resistance in the worm population (Dobson R. J., Le Jambre L. F. & Gill J. H. 1996. Management of anthelmintic resistance: inheritance of resistance and selection with persistent drugs. International Journal for Parasitology. 26: 993-1000).

A second and independent method of increasing efficacy against resistant worms is to combine different action-families into a single product. The underlying principle of this approach is that resistant worms able to survive exposure to one active will be killed by the second (and/or third) active in the mix. The exception is if a single worm carries simultaneously genes for resistance to both drugs. However, if the incidence of resistance is low in a population, the chances of a worm carrying both sets of genes are also very low.

Until recently the only commercially-available anthelmintic products which combined actives against the same parasite species were oral combinations of benzimidazole and levamisole. Substantial formulation problems were inherent in combining the MLs with either of the other two action families—MLs are soluble in oil and require a neutral pH whereas Levamisole is water soluble and requires an acid environment. Benzimidazole actives are insoluble but can be formulated as a stable suspension in water.

This problem has recently been solved with the release of two triple combination products—Triton (described in WO 00/74489, developed by CapTec (a subsidiary of Nufarm) and marketed by Merial) and Erase MC (a mix before use product developed by Coopers). The latter is also available in an ivermectin+Levamisole variant.

All of these products are liquids designed to be dosed orally. The Triton product utilises a suspoemulsion formulation while Erase comes as two liquids which require mixing before use.

However, because these products are administered as a single oral dose and the active agents are usually absorbed and eliminated according to first order kinetic principles, the effective dose rates are only maintained for a short period of time. This short residence time, as outlined previously, results in sub-optimal efficacy against resistant worms.

The threats posed by the developing drug resistance as described above are not restricted to the use of anthelmintic drugs in controlling parasitic infestations in livestock, the development of resistance to drugs used to control a range of animal bacterial and viral infectious diseases having been well documented.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a method of treating animals

characterised by the step of
introducing to the animal a single delivery device containing two or more active agents,
wherein the delivery device is configured to release an effective amount of active agents over a defined time period of 3-14 days.

Preferably the treatment will be formulated to effect a reduction in the parasite burden of an animal, and for ease of reference will be referred to as such throughout the specification. However, this should not be viewed as limiting, for the treatment may alternatively involve the administration of a number of different animal remedies.

For example, it is anticipated that the present invention could be used for treating an animal with an antibiotic, antiviral or antifungal treatment, particularly when attempting to achieve increased efficacy in the treatment of bacterial and viral infectious diseases.

Such treatments may be especially useful in the treatment of animal infections or animal parasites which have developed resistance standard drug treatments.

It is also anticipated that the present invention may find use in other animal treatments, such as the delivery of mineral and/or nutritional supplements, or so forth.

The term “animal” should be taken to encompass any animal in need of a reduction of parasites, including humans. The present invention is particularly suited to production animals, including but not limited to sheep, goats, cattle, deer and pigs.

For ease of reference throughout the present specification, the present invention will be described with reference to sheep, though this should not be seen as a limitation.

In preferred embodiments of the present invention, the active agents will preferably be anthelmintics. For ease of reference throughout the specification, the active agents will be referred to as anthelmintics.

However, this should not be seen as limiting, as the active agents may include antibiotics, antiviral agents, biological agents such as living organisms, micro-organisms and/or reproductive matter thereof, therapeutic substances and/or other drug treatments.

The term “parasite” should be taken to include endoparasites such as helminths, nematodes, cestodes and trematodes; in addition to ectoparasites such as ticks, lice, flies, fleas and so forth.

The term “anthelmintic” should be taken to mean a nematocidal, flukicidal, trematocidal, cestocidal and/or ectoparasiticidal active compound.

The term “effective amount” should be taken to mean the level of anthelmintics necessary to effect a reduction in the level of parasites present in an animal, including a general increase in efficacy against resistant parasites, whilst minimising the undue selection of resistance to anthelmintics and the risk of toxicity to the animal.

In preferred embodiments of the present invention the anthelmintics used may be a macrocyclic lactone such as abamectin and a benzimidazole such as albendazole.

However, once again this should not be seen as limiting and other anthelmintics could be used such as organophosphates, salicylanilide/substituted phenols, tetramisoles or pyrimidine agents. Derivatives and variations of these compounds, and their specific anti-parasitic action are well known in the art and would be known to a skilled addressee.

In preferred embodiments the daily dose may be as close to the normal therapeutic (oral) dose as possible, while minimising toxicity risks. Therefore, for a given weight range of target animal, it is preferable to target the full dose to the lower end of that range.

Preferably, the daily dose delivered is in the order of 3.0-5.0 mg/kg/day of albendazole and 0.1-0.2 mg/kg/day of abamectin.

For example, for adult sheep in the range 50-80 kg it is preferable to deliver approximately 5 mg/kg of albendazole and approximately 0.2 mg/kg of abamectin to the 50 kg animal, which equates to approximately 3.125 mg/kg of albendazole and approximately 0.125 mg/kg of abamectin to the 80 kg animal.

It should be appreciated these dosages are given by way of example only, and should not be viewed as limiting. It is anticipated that the dose rates will vary with different anthelmintics and parasite resistance.

It is anticipated three days will be the minimum required to increase a significant increase in efficacy over standard anthelmintic compositions.

In preferred embodiments of the present invention the defined period of time over which the anthelmintics are released is 5-10 days.

The time period is also a balance between ensuring sufficient duration of exposure to ensure a significant increase in efficacy against resistant worms, whilst minimising the duration of exposure to avoid undue selection for resistance and the risks of toxicity of the anthelmintics to the animal.

It is anticipated that 3-5 days will be the minimum required to ensure a substantial increase in efficacy over traditional anthelmintic preparations.

More preferably, the defined period of time is 6-8 days.

Standard drenching programmes based on periods of weeks are easy to remember and calculate, making the final product user friendly. Further, such a time period allows for some variation in the delivery rates, whilst maintaining the efficacy of the treatment.

There are generally four forms of anthelmintic compositions currently available on the market, a single dose oral liquid drench, a single dose pour-on (dermal) liquid; a single dose liquid injection and sustained controlled release devices which release a low level of anthelmintics from a solid matrix tablet encased in a plastic vehicle over an extended period of time (approximately 100 days).

Oral drenches deliver a one-off high dose of anthelmintic which kills approximately >95% of susceptible nematodes and provides the animal with a short period of time with a low worm burden.

Pour-on and injectable formulations release the drug in response to concentration gradients. This leads to a high initial concentration of drug within the animal, which subsequently declines. Such formulations are no more efficacious than oral formulations and have the disadvantage that the persistent declining concentrations of drug favour the development of resistance (Leathwick and Sutherland 2002 Proceedings of the 32^{nd} Seminar, The Society of Sheep and Beef Cattle Veterinarians, N.Z. Veterinary Association, 115-127). Thus none of these formulations have been shown to have the potential to increase efficacy against resistant worms and/or slow the development of resistance.

It has previously been shown that repeated oral dosing (daily for five days) or continuous intraruminal infusion for a period of five days can increase the efficacy of albendazole treatment.

However, repetitive dosing of extensively-grazed animals with anthelmintics is not practicable. In large animals such as cattle and deer, oral drugs are also extremely difficult to administer, meaning the high majority of farmers use pour-on drenches. As the only combination products on the market are orals, there are very limited options for the use of combination products in these animals.

The available alternative to repetitive dosing is the provision of a controlled release device. A number of studies have shown that efficacy against parasites can be increased by the provision of anthelmintics over an extended period of time, whilst requiring a lower daily level of dose than that required for a single dose.

Some controlled release devices (CRCs) are currently available that release between ⅕^{th} and 1/10^{th} the normal therapeutic dose (depending on animal liveweight) of either albendazole or ivermectin for 100 days. These devices act principally as prophylactics, maintaining parasites at low levels by preventing reinfection. However, such devices typically result in long withholding periods. Recent studies also do not support the view of increased efficacy of these devices over standard oral doses. Furthermore, the prolonged delivery of small doses of anthelmintics may actually select for resistance in the worm population.

Conventional controlled release devices are made from plastic and/or metal components which remain in the animal's rumen. Obviously there is a limit to the number of expired devices that can remain in an animal without consequences for the animal, and thus there is therefore a limit to how many CRCs can be given to an animal. Further, the component residues pose problems in freezing works when the offal is processed.

A second method of increasing efficacy against resistant worms is to combine different action families into a single product. The principle behind this approach is that worms resistant to one active will be killed by the second active. However, until recently the only commercially available anthelmintic products which used actives in combination against the same parasite species were oral combinations of benzimidazole and levamisole. As the anthelmintic classes have differing conditions of solubility and pH, it was difficult to formulate stable compositions of other actives.

If the anthelmintic composition does not kill resistant worms the numbers of such will build up in the population. Therefore, the efficacy of any anthelmintic product against resistant worms is a key feature in delaying the development of severe, production-limiting resistance.

In order to maximise the worm exposure to the anthelmintics, the applicants have developed a method of dosing animals which they have termed “maximum integral dose” and which combines high doses, extended duration and the combination of two or more anthelmintics into a single product, with the aim of achieving extremely high efficacy against parasites, including those resistant to normal doses of single actives.

The integral in mathematics is used to “find the area enclosed by a given curve”—in this case the area under the curve of worm exposure to anthelmintics.

To provide the maximum integral dose, the present invention may preferably utilise a controlled release device that delivers the equivalent of a high oral dose every day for a period of 3-14 days, long enough to provide extremely high efficacy against parasites, but not long enough to build up resistance in the worm population.

The essence of the current invention is to produce a product which delivers enhanced efficacy against most resistant worm genotypes and therefore can be used to delay the emergence of resistance to the constituent actives.

As described above, high dose levels of one or more anthelmintics have previously only been delivered to animals as a single dose, due to the practical difficulties in repetitive dosing of extensively-grazed animals. Therefore, the development of a method for delivering a maximum integral dose from a single delivery device has a number of significant advantages.

In preferred embodiments, the maximum integral dose will be delivered from a short-acting (3-14 day) controlled release device retained in the rumen of an animal by virtue of its density, and which can release multiple anthelmintic actives at a constant, high rate.

By providing a dose rate that is sufficiently high for each active to ensure increased efficacy of each against parasites resistant to that class of drug, it is anticipated that the combination of extended duration with multiple actives will provide a very high efficacy product which will substantially delay the development of resistance.

By way of example, the inventors envisage a three active delivery device containing abamectin, albendazole and tricalbendazole. The first and second actives are nematocidal, while the latter two are flukicides. Such a composition would thus provide two double combinations in a single product containing three actives.

A major impediment to others developing short-acting controlled released devices has thus been the requirement for total degradation of the delivery mechanism. The delivery mechanism of the present invention may preferably be an intraruminal bolus which remains in place due to its density and which degrades completely, leaving no residue in the animal.

A number of intraruminal boluses are known in the art which could be adapted for use in the present invention, such as those described in WO 95/19763 and WO 01/87273.

In some embodiments, two active agents may be incorporated into the core of an intraruminal bolus, with a third active agent in the form of a tablet added to one end of the bolus. In this manner a triple combination of active agents can be delivered—the first as a result of the degradation of the tablet to give an initial dose, followed by the second and third active agents in combination as described above.

Advantages of the present invention include:
1. High efficacy even in the face of moderate levels of resistance
2. Retardation of the further development of resistance
3. Complete degradation of the device, leaving no residue in the animal
4. Combination of otherwise incompatible actives in a solid matrix
5. Although a slow release device, it will not be sufficiently long-acting to pose a serious risk to developing resistance (as with the 100 day CRCs), or require a long withholding period.
6. Although a slow release device, it will deliver doses at or near the same rate as standard oral anthelmintics, significantly higher than other slow release devices, providing greater efficacy.
7. Ability to be used with large animal over 100 kg, due to the ease of use resulting in a viable method of delivering combination products to cattle and deer.

It is thus anticipated that the delivery method may provide increased efficacy against parasites not normally killed effectively by a single oral dose.

Oral albendazole has a label claim for efficacy against adult liver fluke but the level of efficacy appears variable and less than desirable (Coles & Stafford 2001. Veterinary Record 148: 723-724). As in the case of nematodes, extended exposure of flukes to albendazole substantially increases efficacy (Kwan et al., 1988. Journal of Controlled Release 8: 31-38).

Delivery of albendazole through the method of the present invention is anticipated to substantially increase efficacy against adult flukes. Further, while albendazole appears to be ineffective against immature flukes, this may alter the longer-term administration provided by the present invention.

The present invention further provides a composition, delivery means and methods of manufacture thereof, for delivering an effective amount of two or more active agents to animals over a defined period of 3-14 days.

### BRIEF DESCRIPTION OF DRAWINGS

Further aspects of the present invention will become apparent from the following description which is given by way of example only and with reference to the accompanying drawings in which:

FIG. 1 Shows a typical dose/efficacy profile of a single dose of oral anthelmintics;

FIG. 2 Shows a typical dose/efficacy profile of a typical slow release device, and

FIG. 3 Shows a dose/efficacy profile of one preferred embodiment of the present invention.

### BEST MODES FOR CARRYING OUT THE INVENTION

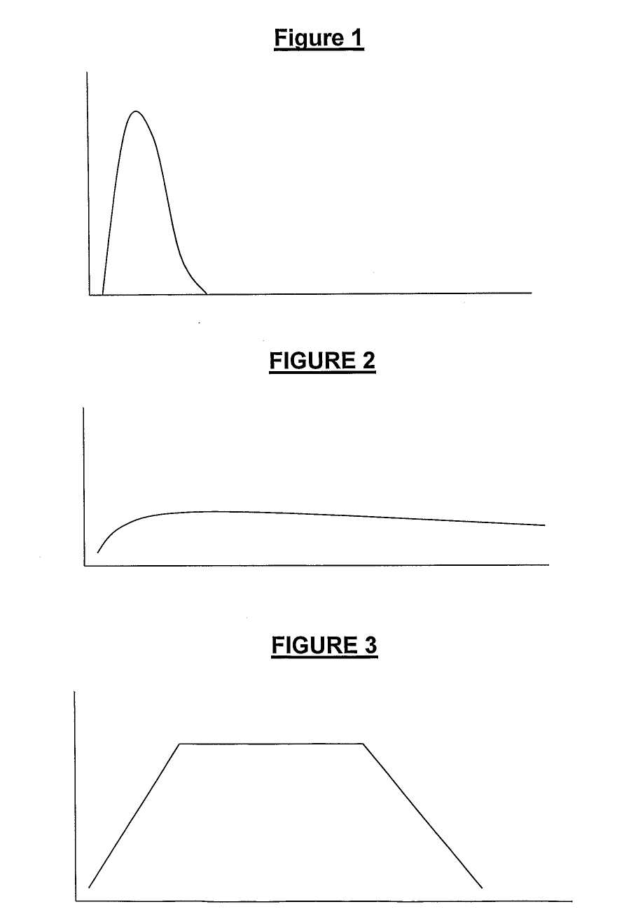

FIG. 1 shows a typical dose/efficacy profile of a single, high dose of an oral anthelmintic. The oral dose removes greater than 95% of susceptible parasites and provides the animal with a period of time with a low parasite burden. However, this is followed by rapid re-infection.

FIG. 2 shows a typical dose/efficacy profile of a current slow release device. These devices deliver a low level of a single anthelmintic over a long period of time (100 days). It acts as a prophylactic, maintaining parasites at low levels. This limits re-infection for about 100 days. Efficacy is similar to that provided by a single oral dose as shown by FIG. 1, but can require a long withholding period after use.

FIG. 3 shows a dose/efficacy profile of one preferred embodiment of the present invention. Parasites are removed at a very high efficacy, with some delayed onset of re-infection and delayed resistance. The extended short duration (3-14 days) also only requires a short withholding period after use.

### Proof of Concept Trials

Two aspects of the present invention which increase efficacy include A) the concept of extended duration and B) the combining of multiple actives; both independently contribute to increasing efficacy.

### A) Extended Duration.

Proof of concept for increasing efficacy with extended duration was demonstrated in two different ways i) trials were conducted using repeated oral dosing to achieve extended duration thereby simulating a controlled release device and ii) Trials were conducted using prototype boluses releasing either albendazole or abamectin, hereinafter referred to as the “Magnum” bolus. Results are given below;

i)—Trials 1 & 2—Repeated Oral Dosing of Albendazole and Abamectin in Lambs to Extend Duration of Exposure.

TABLE 1a & b
Percentage efficacy of albendazole against albendazole
resistant parasites in lambs - efficacy based on worm count.

% reduction in worm count

| 1a - abomasa | Trichostrongylus | Ostertagia |
|---|---|---|
| Treatment | axei | Circumcincta |
| Alb - 5 mg/kg once | 82.9 | 42.7 |
| Alb - 5 mg/kg for | 97.6 | 92.3 |
| 7 days | | |
| Alb - 3.75 mg/kg for | 96.8 | 70.9 |
| 7 days | | |
| Alb - 2.5 mg/kg for | 90.2 | 57.0 |
| 7 days | | |
| Alb - 1.75 mg/kg for | 87.5 | 74.9 |
| 7 days | | |

% reduction in worm count

| 1b - small intestine | Cooperia | Nematodirus |
|---|---|---|
| Treatment | spp. | spp. |
| Alb - 5 mg/kg once | 36.9 | 71.2 |
| Alb - 5 mg/kg for | 86.9 | 73.9 |
| 7 days | | |
| Alb - 3.75 mg/kg for | 83.3 | 88.1 |
| 7 days | | |
| Alb - 2.5 mg/kg for | 67.5 | 69.0 |
| 7 days | | |
| Alb - 1.75 mg/kg for | 57.1 | 58.9 |
| 7 days | | |

TABLE 2
Percentage efficacy of abamectin against abamectin-resistant
parasites in lambs - efficacy based on worm count.

| | % reduction in worm count | | |
|---|---|---|---|
| | Ostertagia | Trichostrongylus | Cooperia |
| Treatment | circumcincta | colubriformis | spp. |
| Aba - 0.2 mg/kg once | 45.0 | 60.7 | 94.9 |
| Aba - 0.18 mg/kg for | 96.4 | 77.1 | 100.0 |
| 7 days | | | |
| Aba - 0.113 mg/kg for | 89.3 | 55.3 | 99.6 |
| 7 days | | | |
| Aba - 0.07 mg/kg for | 77.2 | 47.3 | 99.1 |
| 7 days | | | |
| Moxidectin - 0.2 mg/kg | 76.3 | 72.5 | 99.1 |
| once | | | |

These results showed that against a range of resistant parasites extending the duration of worm exposure to drug was always as good and often far superior to administering a single oral dose. In addition the varying dose rates showed that there was often a benefit in keeping the daily dose rate as high as possible, consistent with the concept of maximum integral dose as outlined in the present specification.

### ii) Trials 3 & 4—Prototype Magnum Boluses Releasing Albendazole in Sheep and Abamectin in Cattle to Achieve Extended Duration of Exposure.

TABLE 3a & b
Percentage efficacy of oral albendazole and a prototype albendazole bolus
against resistant parasites in adult ewes - efficacy based on worm count.

| | % reduction in worm count | | |
|---|---|---|---|
| 3a - abomasa | Ostertagia | Trichostrongylus | Haemonchus |
| Treatment | circumcincta | axei | Contortus |
| Albendazole - 5.0 | 59.3 | 91.8 | 91.4 |
| mg/kg once | | | |
| Albendazole bolus | 86.9 | 92.5 | 98.7 |

| 3b - small intestine | % reduction in worm count |
|---|---|
| Treatment | Cooperia spp. |
| Albendazole - 5.0 | 59.3 |
| mg/kg once | |
| Albendazole bolus | 86.9 |

TABLE 4
Percentage efficacy of pour-on abamectin, pour-on eprinomectin
and a prototype Magnum bolus releasing abamectin against
resistant Cooperia oncophora in cattle

| | % reduction in worm count |
|---|---|
| Treatment | Cooperia oncophora. |
| Abamectin pour-on | 81.0 |
| Eprinomectin pour-on | 83.6 |
| Abamectin bolus | 97.4 |

### B) Combining Actives.

Efficacy data for combining Benzimidazole and Levamisole actives against resistant parasites is reasonably common. Further, there is considerable evidence that these actives work independently and so it can be expected their combined efficacies will work in an additive manner (Anderson et al. 1991. Australian Veterinary Journal 68, 133-136). Hence the expected efficacy of combining two or more actives can be calculated and compared with the measured value.

e.g. Farm 4 from Anderson et al, 1991

| | |
|---|---|
| Efficacy of Levamisole | 88% |
| Efficacy of albendazole | 73% |
| Efficacy of Levamisole + albendazole | 95% |
| Efficacy expected based on additive effects | 97% |

Data on combining the benzimidazole and macrocyclic lactone classes of actives (as is proposed in this specification) is harder to find, but some does exist for goats:

e.g. Data from Pomroy et al, 1992 (New Zealand Veterinary Journal 40, 76-78)

| | |
|---|---|
| Efficacy of ivermectin | 27% |
| Efficacy of oxfendazole | 82% |
| Efficacy of ivermectin + oxfendazole | 97% |
| Efficacy expected based on additive effects | 87% |

This concept has again been tested for the present invention by constructing and testing for efficacy a Magnum bolus releasing 2 actives simultaneously. A prototype sheep bolus designed to release 5 mg/kg of albendazole and 0.18 mg/kg abamectin in a 50 kg sheep was tested against two species of multiple drench resistant parasites. Trial lambs ranged in weight from 48-55.5 kg.

TABLE 5
Efficacy based on worm counts of a Magnum combination bolus
(albendazole + abamectin), single standard oral doses of both
albendazole and abamectin and moxidectin oral against multiple drug
resistant Ostertagia and Trichostrongylus in sheep.

| | Ostertagia | Trichostrongylus |
|---|---|---|
| Treatment group | circumcincta | colubriformis |
| Magnum combination | 99.1 | 100 |
| bolus | | |
| Albendazole oral + | 38.9 | 94.0 |
| abamectin oral | | |
| Moxidectin-oral | 59.9 | 90.6 |

### Conclusion:

The results show a substantial increase in efficacy of the Magnum bolus over a combination of the same actives administered as two single oral doses. This supports the anticipated synergy which is the foundation of the present invention, i.e. extended delivery of each active gives increased efficacy, but by combining multiple actives an even greater step up in efficacy is achieved against resistant worms. This can also been seen in the comparison with moxidectin which although only a single active is recognised as the most potent single active product on the market.

C) Efficacy Against Flukes (Fasciola hepatica).

To test the efficacy of prolonged exposure to albendazole against liver fluke a trial was conducted using an albendazole only bolus in sheep. Sheep from a commercial farm with a previous history of fluke infections were screened by faecal egg count to identify animals carrying fluke infections.

These animals were then randomly allocated to one of two treatment groups on the basis of these egg counts and one group was administered a magnum bolus releasing 5 mg/kg in a 50 kg animal. Twenty days after treatment all animals were slaughtered and livers recovered for fluke counts. Mean numbers of flukes recovered were 12.8 and 2.0 from the control and treated groups respectively, equating to an 84% reduction as a result of treatment.

Thus treatment with the Magnum bolus containing albendazole has resulted in a measureable efficacy against liver flukes.

Aspects of the present invention have been described by way of example only and it should be appreciated that modifications and additions may be made thereto without departing from the scope thereof as defined in the appended claims.

What I/we claim is:

1. A method of treating animals
characterised by the step of
introducing to the animal a single delivery device containing two or more active agents,
wherein the delivery device is configured to release an effective amount of active agents over a defined time period of 3-14 days.

2. A method as claimed in claim 1 wherein the said two or more active agents effect a reduction in the parasite burden of the animal.

3. A method as claimed in claim 1 or claim 2 wherein the said two or more active agents effect a reduction in the number of resistant parasites in the animal.

4. A method as claimed in any one of claims 1 to 3 wherein the said two or more active agents are anthelmintics.

5. A method as claimed in claim 4 wherein said anthelmintics are selected from a list including nematocidal, flukicidal, trematocidal, cestocidal and ectoparasiticidal active agents.

6. A method as claimed in claim 4 or claim 5 wherein said anthelmintics from at least two different action families.

7. A method as claimed in any one of claims 4 to 6 wherein said anthelmintics include a macrocyclic lactone.

8. A method as claimed in claim 7 wherein the macrocyclic lactone is abamectin.

9. A method as claimed in claim 8 wherein the abamectin is delivered at a dosage of substantially 0.1-0.2 mg/kg/day.

10. A method as claimed in any one of claims 4 to 9 wherein said anthelmintics include a benzimidazole.

11. A method as claimed in claim 10 wherein the benzimidazole is albendazole.

12. A method as claimed in claim 11 wherein the albendazole is delivered at a dosage of substantially 3.0-5.0 mg/kg/day.

13. A method as claimed in any one of claims 4 to 12 wherein said anthelmintics include tricalbendazole.

14. A method as claimed in any one of claims 1 to 13 wherein said two or more active agents include at least one antibiotic, antiviral or antifungal agent.

15. A method as claimed in any one of claims 1 to 14 wherein said two or more active agents include biological agents, micro-organisms and/or reproductive matter thereof, therapeutic substances, mineral or nutritional supplements.

16. A method as claimed in any one of claims 1 to 15 wherein the animal is a sheep.

17. A method as claimed in any one of claims 1 to 16 wherein the defined time period is 5-10 days.

18. A method as claimed in any one of claims 1 to 17 wherein the defined time period is 6-8 days.

19. A method as claimed in any one of claims 1 to 18 wherein the parasite is an endoparasite selected from a list including helminths, nematodes, cestodes and trematodes.

20. A method as claimed in any one of claims 1 to 18 wherein the parasite is an ectoparasite selected from a list including ticks, lice, flies and fleas.

21. A method as claimed in any of claims 1 to 20 wherein the delivery device is a controlled release device.

22. A method as claimed in claim 21 wherein the controlled release device is a ruminal bolus.

23. A method as claimed in any one of claims 1 to 22 wherein the delivery device delivers a maximum integral dose.

24. A delivery device for use in a method as claimed in any one of claims 1-23.

25. The use of two or more active agents in the manufacture of a delivery device as claimed in claim 24.

26. A method of treating animals substantially as described herein with reference to and as illustrated by the accompanying description and examples.

27. A delivery device substantially as described herein with reference to and as illustrated by the accompanying description and examples.
